(19) United States
(12) Patent Application Publication
(10) Pub. No.: US 2011/0059985 A1
(43) Pub. Date: Mar. 10, 2011

(54) NOVEL FORMULATION

(76) Inventors: Thomas M. Schmidts, Giessen (DE); Frank Runkel, Giessen (DE)

(21) Appl. No.: 12/739,518

(22) PCT Filed: Oct. 22, 2008
(86) PCT No.: PCT/GB2008/050978

§ 371 Date: Nov. 8, 2010

(30) Foreign Application Priority Data
Oct. 23, 2007 (GB) 0720716.0

(51) Int. Cl. A61K 31/496; A61K 47/32; A61K 47/34; A61K 31/506; A61K 31/4418; A61P 31/10; A61P 31/04; A61P 31/12; A61P 17/00; A61P 43/00

(52) U.S. Cl. 514/254.07; 514/772.1; 514/772; 514/275; 514/345

(57) ABSTRACT

The present invention relates to a pharmaceutical formulation comprising a pharmaceutically activeagent; water; a polyethylene glycol or a poloxamer; and a polyethylene glycol mono- or di-ether. Preferably the pharmaceutically active agent is an anti-fungal or anti-mycotic agent. Preferably the pharmaceutically active agent is lipophilic and/or keratinophilic. The present invention also relates to the use of the formulation in treating diseases, disorders or pathological conditions of the nail or skin, such as onychomycosis, dermatomycosis and other mycoses. The present invention also relates to a method of administering a pharmaceutically active agent to a subject by applying the formulation comprising the pharmaceutically active agent to a nail or skin of the subject. The present invention further relates to a method of preparing the formulation.

### FIELD OF THE INVENTION

The present invention relates to a pharmaceutical formulation comprising a pharmaceutically active agent; water; a polyethylene glycol or a poloxamer; and a polyethylene glycol mono- or di-ether. Preferably the pharmaceutically active agent is an anti-fungal or anti-mycotic agent. Preferably the pharmaceutically active agent is lipophilic and/or keratinophilic. The present invention also relates to the use of the formulation in treating diseases, disorders or pathological conditions of the nail or skin, such as onychomycosis, dermatomycosis and other mycoses. The present invention also relates to a method of administering a pharmaceutically active agent to a subject by applying the formulation comprising the pharmaceutically active agent to a nail or skin of the subject. The present invention further relates to a method of preparing the formulation.

### BACKGROUND OF THE INVENTION

Although diseases and disorders of the skin can often be treated effectively by topical administration of pharmaceutically active agents, successful treatment of diseases and disorders of the nails has remained elusive. It has proven difficult to deliver pharmaceutically active agents effectively into and beneath the nails where the cause of most pathological conditions of the nails originates.

In particular fungal infections of the nails remain ineffectively treated. Fungal infections in, under and around fingernails and toenails are generally referred to as onychomycosis. Onychomycosis is most frequently caused by dermatophytes such as Trichophyton rubrum, Trichophyton mentagrophytes and Epidermophyton floccosum, but can also be caused by other types of fungi including moulds, yeasts and the like. Onychomycosis that is not caused by dermatophytes is normally caused by Candida species. Mixed infections can also occur.

Onychomycosis causes thickening, roughness, splitting and discolouration of the nail and can even result in its loss or destruction. In addition, it can be the cause of pain, inadequate blood supply, problems with walking, and other undesirable phenomena.

In the past, onychomycosis was treated inter alia by removing the affected part of the nail or the whole nail. However, this type of treatment can lead to permanent damage to the nail. Also, the newly growing nail can grow in a misshapen form. Moreover, there is no guarantee that the onychomycosis can be completely cured by removing the nail.

Instead of removing the nail, onychomycosis can also be treated by the use of various anti-mycotic agents. The anti-mycotic agents can be administered orally, for example. In this form of treatment, however, stress is put on the body as a whole and only a small amount of the anti-mycotically active substance reaches the nail via the nail matrix. Oral treatment has the further disadvantage that such treatment requires a treatment time of at least 12 weeks for toenails and about 6 to 8 weeks for fingernails. Such long treatment times make the treatment expensive and reduce patient compliance. Furthermore, oral treatment increases the risk of side-effects, such as, for example, irritation of the gastro-intestinal tract, nausea, undesirable interactions with other medicaments, active ingredient induced skin rashes etc. The oral treatment of onychomycosis is further rendered difficult by variable rates of absorption and metabolism.

Another method of treating onychomycosis comprises the topical application of a pharmaceutical formulation containing an anti-mycotic active ingredient. For example, it is known to treat onychomycosis with nail lacquer formulations that contain an anti-mycotic active ingredient. However, such anti-fungal nail lacquers lack the necessary penetrating power to reach the fungal infection, because the nail is a difficult barrier for the anti-fungal compounds to penetrate.

Accordingly, there remains a need for the effective treatment of diseases, disorders and pathological conditions of the nail such as onychomycosis. It would be advantageous to have a topical formulation that is capable of penetrating the nail barrier and capable of effectively treating nail fungal diseases, thus avoiding oral administration of anti-fungal agents and the necessity of removing the nail. To be effective, a topical treatment for onychomycosis should exhibit a powerful potency for pathogens and must be able to permeate through the nail barrier.

### SUMMARY OF THE INVENTION

Accordingly, a first aspect of the present invention provides a formulation comprising:

(a) a pharmaceutically active agent;

(b) water;

(c) a polyethylene glycol (PEG) or a poloxamer; and

(d) a polyethylene glycol mono- or di-ether.

A polyethylene glycol (PEG) has the general formula HO—(CH₂CH₂O)ₙ—H. Preferably n=4-2000, preferably n=6-750, preferably n=150-500. In a preferred embodiment, the polyethylene glycol has a mean molecular weight of at least 400, preferably at least 500, preferably at least 700, preferably at least 1000, preferably at least 1500, preferably at least 4500, preferably at least 5000, preferably at least 6000, and more preferably at least 8000. Preferably the mean molecular weight of the polyethylene glycol is no more than 100000, preferably no more than 30000, and more preferably no more than 20000. Any of these preferred lower molecular weight limits can be combined with any of these preferred upper molecular weight limits to give preferred molecular weight ranges. Preferably the mean molecular weight of the polyethylene glycol is in the range of 200-100000, preferably in the range of 300-30000. In a preferred embodiment, the polyethylene glycol is PEG 8000-20000, i.e. a polyethylene glycol having a mean molecular weight between 8000 and 20000. In an alternate preferred embodiment, the mean molecular weight of the polyethylene glycol is in the range of 200-600, preferably in the range of 300-500, and more preferably the mean molecular weight of the polyethylene glycol is about 400. In a preferred embodiment, the formulation comprises the polyethylene glycol in an amount of 5-50%, preferably in an amount of 10-40%, preferably in an amount of 15-35%.

For the purposes of the present invention, unless stated otherwise all amount percentages refer to the percentage by weight.

A poloxamer is a polyethylene glycol-polypropylene glycol block copolymer with the general formula HO—(CH₂CH₂O)ₐ—(CH(CH₃)CH₂O)_{b}—(CH₂CH₂O)_{c}—H. Preferably a=4-200. Preferably b=15-350. Preferably c=4-200. Preferably the polyoxyethylene content of the poloxamer is 10-80% of the total polymer weight.

In a preferred embodiment, the poloxamer has a mean molecular weight of at least 1000, preferably at least 2000, preferably at least 4500, preferably at least 5000, preferably at least 6000, and more preferably at least 8000. Preferably the mean molecular weight of the poloxamer is no more than 100000, preferably no more than 30000, and more preferably no more than 15000. Any of these preferred lower molecular weight limits can be combined with any of these preferred upper molecular weight limits to give preferred molecular weight ranges. Preferably the mean molecular weight of the poloxamer is in the range of 1000-16000, preferably in the range of 2000-15000. In a preferred embodiment, the formulation comprises the poloxamer in an amount of at least 1%, preferably at least 2%, preferably at least 5%. Preferably the formulation comprises the poloxamer in an amount of 5-50%, preferably in an amount of 10-40%, preferably in an amount of 15-35%.

The formulation of the present invention may comprise a polyethylene glycol or a poloxamer. Preferably the formulation comprises a polyethylene glycol. In one embodiment, the formulation does not comprise a poloxamer.

A polyethylene glycol mono- or di-ether has the general formula RO—(CH₂CH₂O)ₘ—R. Preferably m=2-250, preferably m=4-175, preferably m=6-125. Preferably each R is independently selected from hydrogen or an optionally substituted alkyl, alkenyl, alkynyl, aryl, arylalkyl, arylalkenyl, arylalkynyl, alkylaryl, alkenylaryl or alkynylaryl group; more preferably each R is independently selected from hydrogen or an optionally substituted alkyl, aryl, arylalkyl or alkylaryl group; more preferably each R is independently selected from hydrogen or an optionally substituted alkyl group; more preferably each R is independently selected from hydrogen or a methyl or ethyl group; all provided that at least one R is not hydrogen. In a preferred embodiment, one R is hydrogen. Preferably R is not substituted. Preferably R comprises no heteroatoms in its carbon skeleton. Preferably R contains from 1 to 20 carbon atoms, preferably from 1 to 15 carbon atoms, preferably from 1 to 10 carbon atoms, preferably from 1 to 5 carbon atoms, more preferably from 1 to 4 carbon atoms. Preferably the polyethylene glycol mono- or di-ether contains a single —(CH₂CH₂O)ₘ— group, i.e. no R comprises a —(CH₂CH₂O)ₘ— group. Preferably the mean molecular weight of the polyethylene glycol mono- or di-ether is in the range of 120-10000, preferably in the range of 200-8000, preferably in the range of 300-5000. In a preferred embodiment, the formulation comprises the polyethylene glycol mono- or di-ether in an amount of 0.1-30%, preferably in an amount of 2-15%, preferably in an amount of 3-10%, more preferably in an amount of about 5%. In an alternative preferred embodiment, the formulation comprises the polyethylene glycol mono- or di-ether in an amount of 4-30%, preferably in an amount of 4-20%, more preferably in an amount of about 5%.

Preferably the formulation comprises a polyethylene glycol mono-ether.

In one embodiment, the formulation comprises a polyethylene glycol di-ether, preferably wherein each R independently contains from 1 to 20 carbon atoms, preferably from 1 to 15 carbon atoms, preferably from 1 to 10 carbon atoms, preferably from 1 to 5 carbon atoms, more preferably from 1 to 4 carbon atoms.

In a preferred embodiment, the polyethylene glycol mono- or di-ether is a polyethylene glycol mono- or di-methyl or ethyl ether, more preferably the polyethylene glycol mono- or di-ether is polyethylene glycol monomethyl ether (MPEG). Preferably the polyethylene glycol monomethyl ether is MPEG 350-10000, i.e. a polyethylene glycol monomethyl ether having a mean molecular weight between 350 and 10000. More preferably, the polyethylene glycol monomethyl ether is MPEG 350-5000, i.e. a polyethylene glycol monomethyl ether having a mean molecular weight between 350 and 5000. Preferably, the polyethylene glycol monomethyl ether is MPEG 2000, i.e. a polyethylene glycol monomethyl ether having a mean molecular weight of about 2000. In a preferred embodiment, the formulation comprises polyethylene glycol monomethyl ether in an amount of 2-15%, preferably in an amount of 3-10%.

Preferably the polyethylene glycol (PEG) or poloxamer on the one hand and the polyethylene glycol mono- or di-ether on the other hand are used in a ratio of at least 1:1, preferably at least 2:1, more preferably at least 3:1. Preferably the polyethylene glycol (PEG) or poloxamer on the one hand and the polyethylene glycol mono- or di-ether on the other hand are used in a ratio of no more than 10:1, preferably no more than 8:1, more preferably no more than 6:1. Any of these preferred lower ratios can be combined with any of these preferred upper ratios to give preferred ratio ranges. Preferably the polyethylene glycol (PEG) or poloxamer on the one hand and the polyethylene glycol mono- or di-ether on the other hand are used in a ratio of from 10:1 to 1:1, preferably in a ratio of about 4:1.

For the purposes of the present invention, an ‘alkyl’ group is defined as a monovalent saturated hydrocarbon, which may be straight-chained or branched, or be or include cyclic groups. An alkyl group may optionally include one or more heteroatoms N, O or S in its carbon skeleton. Examples of alkyl groups are methyl, ethyl, n-propyl, i-propyl, n-butyl, i-butyl, t-butyl and n-pentyl groups. Preferably an alkyl group is straight-chained or branched and does not include any heteroatoms in its carbon skeleton. Preferably an alkyl group is a C₁-C₁₂ alkyl group, which is defined as an alkyl group containing from 1 to 12 carbon atoms. More preferably an alkyl group is a C₁-C₆ alkyl group, which is defined as an alkyl group containing from 1 to 6 carbon atoms. An ‘alkylene’ group is similarly defined as a divalent alkyl group.

An ‘alkenyl’ group is defined as a monovalent hydrocarbon, which comprises at least one carbon-carbon double bond, which may be straight-chained or branched, or be or include cyclic groups. An alkenyl group may optionally include one or more heteroatoms N, O or S in its carbon skeleton. Examples of alkenyl groups are vinyl, allyl, but-1-enyl and but-2-enyl groups. Preferably an alkenyl group is straight-chained or branched and does not include any heteroatoms in its carbon skeleton. Preferably an alkenyl group is a C₂-C₁₂ alkenyl group, which is defined as an alkenyl group containing from 2 to 12 carbon atoms. More preferably an alkenyl group is a C₂-C₆ alkenyl group, which is defined as an alkenyl group containing from 2 to 6 carbon atoms. An ‘alkenylene’ group is similarly defined as a divalent alkenyl group.

An ‘alkynyl’ group is defined as a monovalent hydrocarbon, which comprises at least one carbon-carbon triple bond, which may be straight-chained or branched, or be or include cyclic groups. An alkynyl group may optionally include one or more heteroatoms N, O or S in its carbon skeleton. Examples of alkynyl groups are ethynyl, propargyl, but-1-ynyl and but-2-ynyl groups. Preferably an alkynyl group is straight-chained or branched and does not include any heteroatoms in its carbon skeleton. Preferably an alkynyl group is a C₂-C₁₂ alkynyl group, which is defined as an alkynyl group containing from 2 to 12 carbon atoms. More preferably an alkynyl group is a C₂-C₆ alkynyl group, which is defined as an alkynyl group containing from 2 to 6 carbon atoms. An ‘alkynylene’ group is similarly defined as a divalent alkynyl group.

An ‘aryl’ group is defined as a monovalent aromatic hydrocarbon. An aryl group may optionally include one or more heteroatoms N, O or S in its carbon skeleton. Examples of aryl groups are phenyl, naphthyl, anthracenyl and phenanthrenyl groups. Preferably an aryl group does not include any heteroatoms in its carbon skeleton. Preferably an aryl group is a C₄-C₁₄ aryl group, which is defined as an aryl group containing from 4 to 14 carbon atoms. More preferably an aryl group is a C₆-C₁₀ aryl group, which is defined as an aryl group containing from 6 to 10 carbon atoms. An ‘arylene’ group is similarly defined as a divalent aryl group.

For the purposes of the present invention, where a combination of groups is referred to as one moiety, for example, arylalkyl, arylalkenyl, arylalkynyl, alkylaryl, alkenylaryl or alkynylaryl, the last mentioned group contains the atom by which the moiety is attached to the rest of the molecule. A typical example of an arylalkyl group is benzyl.

For the purposes of this invention, an optionally substituted alkyl, alkenyl, alkynyl, aryl, arylalkyl, arylalkenyl, arylalkynyl, alkylaryl, alkenylaryl or alkynylaryl group may be substituted with one or more of —F, —Cl, —Br, —I, —CF₃, —CCl₃, —CBr₃, —CI₃, —OH, —SH, —NH₂, —CN, —NO₂, —COOH, —R^{α}—O—R^{β}, —R^{α}—S—R^{β}, —R^{α}—SO—R^{β}, —R^{α}—SO₂—R^{β}, —R^{α}—SO₂—OR^{β}, —R^{α}O—SO₂—R^{β}, —R^{α}—SO₂—N(R^{β})₂, —R^{α}—NR^{β}—SO₂—R^{β}, —R^{α}O—SO₂—OR^{β}, —R^{α}O—SO₂—N(R^{β})₂, —R^{α}—NR^{β}—SO₂—OR^{β}, —R^{α}—NR^{β}—SO₂—N(R^{β})₂, —R^{α}—N(R^{β})₂, —R^{α}—N(R^{β})₃⁺, —R^{α}—P(R^{β})₂, —R^{α}—Si(R^{β})₃, —R^{α}—CO—R^{β}, —R^{α—CO—OR}^{β}, —R^{α}O—CO—R^{β}, —R^{α}—CO—N(R^{β})₂, —R^{α}—NR^{β}—CO—R^{β}, —^{α}O—CO—OR^{β}, —R^{α}O—CO—N(R^{β})₂, —R^{α}—NR^{β}—CO—OR^{β}, —R^{α}—NR^{β}—CO—N(R^{β})₂, —R^{α}—CS—R^{β}, —R^{α}—CS—OR^{β}, —R^{α}O—CS—R^{β}, —R^{α}—CS—N(R^{β})₂, —R^{α}—NR^{β}—CS—R^{β}, —R^{α}O—CS—OR^{β}, —R^{α}O—CS—N(R^{β})₂, —R^{α}—NR^{β}—CS—OR^{β}, —R^{α}—NR^{β}—CS—N(R^{β})₂, —R^{β}a bridging substituent such as —O—, —S—, —NR^{β}— or —R^{α}—, or a π-bonded substituent such as ═O, —S or ═NR^{β}. In this context, —R^{α}— is independently a chemical bond, a C₁-C₁₀ alkylene, C₁-C₁₀ alkenylene or C₁-C₁₀ alkynylene group. —R^{β} is independently hydrogen, unsubstituted C₁-C₆ alkyl or unsubstituted C₆-C₁₀ aryl. Optional substituent(s) are taken into account when calculating the total number of carbon atoms in the parent group substituted with the optional substituent(s). Preferably an optionally substituted alkyl, alkenyl, alkynyl, aryl, arylalkyl, arylalkenyl, arylalkynyl, alkylaryl, alkenylaryl or alkynylaryl group is not substituted with a bridging substituent. Preferably an optionally substituted alkyl, alkenyl, alkynyl, aryl, arylalkyl, arylalkenyl, arylalkynyl, alkylaryl, alkenylaryl or alkynylaryl group is not substituted with a π-bonded substituent. Preferably a substituted group comprises 1, 2 or 3 substituents, more preferably 1 or 2 substituents, and even more preferably 1 substituent.

Any optional substituent may be protected. Suitable protecting groups for protecting optional substituents are known in the art, for example from ‘Protective Groups in Organic Synthesis’ by T. W. Greene and P. G. M. Wuts (Wiley-Interscience, 4^{th} edition, 2006).

In a preferred embodiment, the formulation comprises:

(a) a pharmaceutically active agent;

(b) water;

(c) polyethylene glycol (PEG); and

(d) polyethylene glycol monomethyl ether (MPEG).

In another preferred embodiment, the formulation comprises:

(a) 0.1-30% pharmaceutically active agent;

(b) 5-50% water;

(c) 5-50% polyethylene glycol; and

(d) 2-15% polyethylene glycol monomethyl ether;

and also optionally:

(e) 0-70% alcohol;

(f) 0-5% acid or base for pH adjustment;

(g) 0-10% penetration enhancer; and

(h) 0-6% plasticizer.

In another preferred embodiment, the formulation comprises:

(a) 0.1-30% pharmaceutically active agent;

(b) 5-50% water;

(c) 5-50% polyethylene glycol; and

(d) 2-15% polyethylene glycol monomethyl ether;

and also optionally:

(e) 0-70% alcohol;

(f) 0-5% acid or base for pH adjustment;

(g) 0-1% isopropyl myristate;

(h) 0-4% transcutol; and

(i) 0-5% propylene glycol.

In a preferred embodiment, the pharmaceutically active agent is an anti-fungal or anti-mycotic agent. The terms ‘anti-fungal’ and ‘anti-mycotic’ are used interchangeable herein. Preferably the pharmaceutically active agent is lipophilic and/or keratinophilic.

In another preferred embodiment, the anti-fungal or anti-mycotic agent is an azole, imidazole, triazole, thiazole, thiadiazole, guanidine, pyrimidine, imine, morpholine, 2-pyridone, 2-pyrimidone, allylamine, benzylamine, polyene, echinocandin, benzofuran, benzoxaborole, pyridine, or thiocarbamate. If the anti-fungal or anti-mycotic agent is an imidazole, then it is preferably bifonazole, clotrimazole, econazole, fenticonazole, isoconazole, ketoconazole, miconazole, oxiconazole, tioconazole, sertaconazole, sulconazole, or a pharmaceutically acceptable salt thereof. If the anti-fungal or anti-mycotic agent is a triazole, then it is preferably fluconazole, itraconazole, posaconazole, ravuconazole, terconazole, voriconazole, or a pharmaceutically acceptable salt thereof. If the anti-fungal or anti-mycotic agent is a thiazole, then it is preferably a 2-amino-thiazole, preferably abafungin or a pharmaceutically acceptable salt thereof. If the anti-fungal or anti-mycotic agent is a guanidine, then it is preferably an arylguanidine, preferably abafungin or a pharmaceutically acceptable salt thereof. If the anti-fungal or anti-mycotic agent is a pyrimidine, then it is preferably a 2-pyrimidinimine, preferably abafungin or a pharmaceutically acceptable salt thereof. If the anti-fungal or anti-mycotic agent is an imine, then it is preferably a 2-pyrimidinimine, preferably abafungin or a pharmaceutically acceptable salt thereof. If the anti-fungal or anti-mycotic agent is a morpholine, then it is preferably amorolfine or a pharmaceutically acceptable salt thereof. If the anti-fungal or anti-mycotic agent is a 2-pyridone, then it is preferably ciclopirox or a pharmaceutically acceptable salt thereof. If the anti-fungal or anti-mycotic agent is a 2-pyrimidone, then it is preferably flucytosine or a pharmaceutically acceptable salt thereof. If the anti-fungal or anti-mycotic agent is an allylamine, then it is preferably terbinafine, naftifine, or a pharmaceutically acceptable salt thereof. If the anti-fungal or anti-mycotic agent is a benzylamine, then it is preferably butenafine or a pharmaceutically acceptable salt thereof. If the anti-fungal or anti-mycotic agent is a polyene, then it is preferably amphotericin B, nystatin, pimaricin (also called natamycin), or a pharmaceutically acceptable salt thereof. If the anti-fungal or anti-mycotic agent is an echinocandin, then it is preferably caspofungin, micafungin, anidulafungin, or a pharmaceutically acceptable salt thereof. Preferably the anti-fungal or anti-mycotic agent is abafungin or a pharmaceutically acceptable salt thereof, preferably abafungin.

For the purposes of the present invention, if a compound is said to be an azole, imidazole, triazole, thiazole, 2-amino-thiazole, thiadiazole, guanidine, arylguanidine, pyrimidine, imine, 2-pyrimidinimine, morpholine, 2-pyridone, 2-pyrimidone, allylamine, benzylamine, polyene, echinocandin, benzofuran, benzoxaborole, pyridine, thiocarbamate etc, then this means that the compound comprises an azole, imidazole, triazole, thiazole, 2-amino-thiazole, thiadiazole, guanidine, arylguanidine, pyrimidine, imine, 2-pyrimidinimine, morpholine, 2-pyridone, 2-pyrimidone, allylamine, benzylamine, polyene, echinocandin, benzofuran, benzoxaborole, pyridine, thiocarbamate etc functional group.

Azoles are generally considered to be five-membered aromatic heterocycles comprising one nitrogen atom and at least one further heteroatom, such as a nitrogen, oxygen or sulphur atom. Therefore imidazoles (five-membered aromatic heterocycles comprising two nitrogen atoms), triazoles (five-membered aromatic heterocycles comprising three nitrogen atoms), thiazoles (five-membered aromatic heterocycles comprising one nitrogen atom and one sulphur atom), and thiadiazoles (five-membered aromatic heterocycles comprising two nitrogen atoms and one sulphur atom) are generally considered to be azoles.

However, when referring to azole anti-fungal agents, generally only imidazole and triazole anti-fungal agents are meant, not thiazole or thiadiazole anti-fungal agents. Without wishing to be bound by theory, this is because currently the anti-fungal activity of imidazole and triazole anti-fungal agents is believed to be due to the inhibition of the ergosterol biosynthesis by inhibiting 14α-demethylase. Thiazole anti-fungal agents, on the other hand, are currently not believed to inhibit 14α-demethylase and their anti-fungal activity is currently believed to be at least partially due to the inhibition of the ergosterol biosynthesis by inhibiting 24-sterolmethyltransferase.

Therefore, for the purposes of the present invention, the term ‘azole’ encompasses all five-membered aromatic heterocycles comprising one nitrogen atom and at least one further heteroatom, and therefore includes imidazoles, triazoles, thiazoles, and thiadiazoles. In a preferred embodiment, the term ‘azole’ only encompasses imidazoles and triazoles.

In one embodiment of the present invention, the anti-fungal or anti-mycotic agent is not a triazole. In another embodiment, the anti-fungal or anti-mycotic agent is not an imidazole. In another embodiment, the anti-fungal or anti-mycotic agent is a thiazole or a thiadiazole.

In a preferred embodiment of the present invention, the anti-fungal or anti-mycotic agent is a compound of the general formula (I):

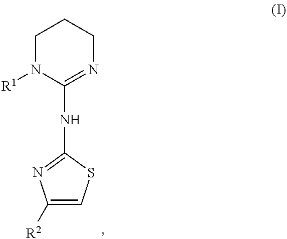

wherein
R¹ is hydrogen or alkyl; and
R² is a group of the formula:

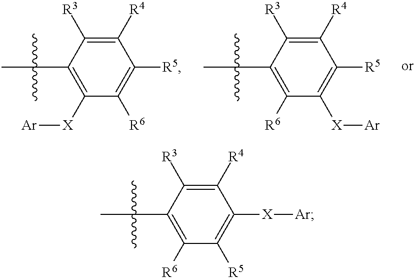

wherein
R³, R⁴, R⁵ and R⁶ are independently hydrogen, halogen, nitro, alkyl, alkoxy, alkoxy-carbonyl, dialkylamino, alkylthio, alkylsulphinyl, alkylsulphonyl, haloalkyl, haloalkoxy, haloalkylthio, haloalkylsulphinyl, or haloalkylsulphonyl;
X is oxygen, sulphur, sulphinyl, or sulphonyl; and
Ar is an optionally substituted aryl group;
   or a pharmaceutically acceptable salt thereof.

The compounds of formula (I) are in equilibrium with their tautomers of formulae (Ia) and (Ib):

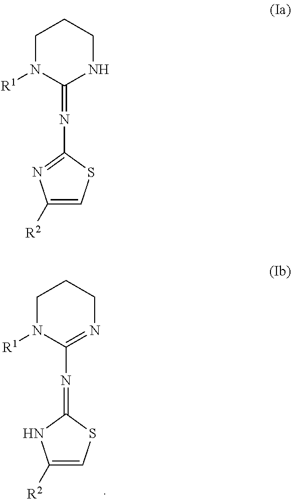

Preferably R¹ is hydrogen or C₁₋₃ alkyl, preferably hydrogen. Preferably R³, R⁴, R⁵ and R⁶ are independently hydrogen or C₁₋₃ alkyl, preferably hydrogen. Preferably X is oxygen. Preferably Ar is a phenyl group optionally substituted with one, two or three C₁₋₃ alkyl or C₁₋₃ alkoxy groups. Preferably R² is:

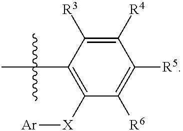

The compounds of formula (I) can be classified as being 2-amino-thiazoles, or arylguanidines, or 2-pyrimidinimines.

A preferred compound of the general formula (I) is abafungin of the formula (II):

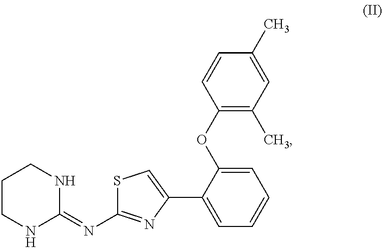

which is in equilibrium with its tautomers of formulae (IIa) and (IIb):

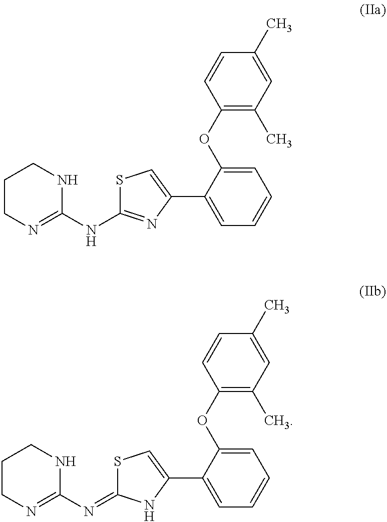

In another preferred embodiment, the anti-fungal or anti-mycotic agent is abafungin, ciclopirox olamine, terbinafine hydrochloride, or amorolfine. Preferably the anti-fungal or anti-mycotic agent is abafungin or a pharmaceutically acceptable salt thereof, preferably abafungin. If the anti-fungal or anti-mycotic agent is abafungin, the formulation preferably further comprises an acid such as formic acid for pH adjustment. Preferably the formulation has a pH in the range of about 5-8, preferably about 5-7, preferably about 5-6, preferably about 5.5, which simulates the conditions of human skin and nails. In an alternate embodiment, the formulation has a pH in the range of about 1-7, preferably about 2-6, preferably about 3-6, preferably about 3-5, more preferably about 4-5.

In a preferred embodiment, the formulation comprises the pharmaceutically active agent in an amount of 0.1-30%, preferably in an amount of 0.5-20%, preferably in an amount of 1-15%. Preferably the formulation comprises the pharmaceutically active agent in an amount of at least 2.5%, preferably at least 4%, preferably at least 5%, more preferably in an amount of about 10%.

In a preferred embodiment, the pharmaceutically active agent is substantially dissolved in the formulation, i.e. at least 75% of the pharmaceutically active agent present in the formulation is in solution in the formulation. Preferably at least 90%, preferably at least 95%, preferably at least 98%, preferably at least 99%, more preferably at least 99.9% of the pharmaceutically active agent present in the formulation is in solution in the formulation.

In a preferred embodiment, the formulation comprises water in an amount of 5-50%, preferably in an amount of 10-50%, preferably in an amount of 17-25% or 20-40%. More preferably the formulation comprises water in an amount of about 20%.

In a preferred embodiment, the formulation further comprises an alcohol, such as 2-propanol, ethanol, benzyl alcohol, or 2-phenoxyethanol. The formulation may comprise up to 70% alcohol. If the formulation comprises an alcohol, it is preferably present in an amount of 10-70%, preferably in an amount of 20-60%, preferably in an amount of 30-50%.

In some embodiments, the formulation further comprises an acid or a base for pH adjustment. Suitable acids include organic fatty acids which may be saturated or unsaturated (such as citric acid, myristic acid and formic acid) and inorganic acids (such as hydrochloric acid and sulphuric acid). A preferred acid is formic acid. Suitable bases include sodium hydroxide. The formulation may comprise up to 5% acid or base. Preferably the formulation has a pH in the range of about 5-8, preferably about 5-7, preferably about 5-6, preferably about 5.5, which simulates the conditions of human skin and nails. In an alternate embodiment, the formulation has a pH in the range of about 1-7, preferably about 2-6, preferably about 3-6, preferably about 3-5, more preferably about 4-5.

In a preferred embodiment, the formulation further comprises a penetration enhancer and/or a plasticizer. Preferred penetration enhancers and/or plasticizers include, but are not limited to isopropyl myristate, transcutol, propylene glycol, isopropyl palmitate, terpenoides, decyl oleate, oleic acid, sulphoxides, keratinolytics (such as urea), azones, terpenes, essential oils, surfactants (such as Tween 20, Tween 80, Span, Labrasol, Isoceteth-20), alcohols, polyols, fatty acids, glycols, and pyrrolidones. The formulation may comprise up to 10% penetration enhancer preferably up to 6%. The formulation may comprise up to 6% plasticizer, preferably up to 5%.

In a preferred embodiment, the formulation comprises isopropyl myristate. The formulation may comprise up to 1% isopropyl myristate. If the formulation comprises isopropyl myristate, it is preferably present in an amount of 0.1-1%, preferably 0.5-1%.

In a preferred embodiment, the formulation comprises a penetration enhancer such as transcutol. The formulation may comprise up to 4% transcutol. If the formulation comprises transcutol, it is preferably present in an amount of 0.5-4%, preferably in an amount of 1-4%, preferably in an amount of 2-4%.

In a preferred embodiment, the formulation comprises propylene glycol. The formulation may comprise up to 5% propylene glycol. If the formulation comprises propylene glycol, it is preferably present in an amount of 0.5-5%, preferably in an amount of 0.5-4%, preferably in an amount of 0.5-3%.

In a preferred embodiment, the formulation has a viscosity of at least 1100 mPas, preferably at least 1200 mPas, preferably at least 1300 mPas, preferably at least 1500 mPas, preferably at least 2000 mPas, preferably at least 5000 mPas, preferably at least 10000 mPas. In an alternate preferred embodiment, the formulation has a viscosity of between 2 and 1000 mPas, preferably between 5 and 900 mPas, preferably between 10 and 750 mPas, preferably between 30 and 500 mPas.

In a particularly preferred embodiment the formulation has a viscosity of between 100 and 500 mPas, preferably between 200 and 300 mPas, more preferably about 250 mPas. Preferably such a formulation is suitable for application to the nail, preferably as a gel.

In another particularly preferred embodiment the formulation has a viscosity of between 30 and 100 mPas, preferably between 40 and 80 mPas, more preferably about 60 mPas. Preferably such a formulation is suitable for application to the skin, preferably as a spray.

In a preferred embodiment, the formulation is not a solid. Preferably the formulation is a spray, cream, ointment, gel or paste. More preferably the formulation is a hydrophilic water-based gel.

The formulation of the present invention can be used for the treatment of a disease, disorder or pathological condition of the nail or skin. For example, the formulation of the present invention can be used for the treatment of onychomycoses, dermatomycoses, oral, vaginal or anal mycoses, skin diseases such as acne, topical bacterial infections such as Staphylococcus aureus, or topical viral infections such as herpes. The formulation of the present invention can also be used to aid wound healing.

Accordingly, it is preferred that the formulation of the present invention is suitable for topical application, preferably to the nail or skin.

Alternatively the formulation of the present invention can be used for the treatment of a disease, disorder or pathological condition of the hooves, horn, claws or skin of a subject, preferably a non-human mammal such as a cow, pig, sheep, dog or cat. Preferably the disease, disorder or pathological condition is a fungal infection.

A second aspect of the present invention provides a method of administering a pharmaceutically active agent to a subject, comprising applying a formulation according to the first aspect of the present invention to a nail of the subject. Preferably the pharmaceutically active agent is lipophilic and/or keratinophilic.

Lipophilic and/or keratinophilic pharmaceutically active agents are often capable of penetrating skin. Hydrophilic pharmaceutically active agents are often capable of penetrating nails. The method of the second aspect of the present invention uses a hydrophilic formulation to make it possible for lipophilic and/or keratinophilic pharmaceutically active agents to penetrate nails.

Preferably the subject is a human or non-human mammal, preferably a human. Preferably the pharmaceutically active agent penetrates into the subject's nail and nail matrix by penetrating through the nail and through the skin surrounding the nail.

A third aspect of the present invention provides a method of treating onychomycosis, the method comprising applying a formulation according to the first aspect of the present invention to the nail of a subject suffering from onychomycosis.

The third aspect of the present invention also provides a method of treating dermatomycosis, the method comprising applying a formulation according to the first aspect of the present invention to the skin of a subject suffering from dermatomycosis.

The third aspect of the present invention further provides a method of treating an oral, vaginal or anal mycosis, the method comprising applying a formulation according to the first aspect of the present invention to the skin or mucosa of a subject suffering from the oral, vaginal or anal mycosis.

The third aspect of the present invention further provides a method of treating a skin disease (such as acne), the method comprising applying a formulation according to the first aspect of the present invention to the skin of a subject suffering from the skin disease.

The third aspect of the present invention further provides a method of treating a topical bacterial infection (such as Staphylococcus aureus) or a topical viral infection (such as herpes), the method comprising applying a formulation according to the first aspect of the present invention to the skin or mucosa of a subject suffering from the topical infection.

The third aspect of the present invention further provides a method of aiding wound healing, the method comprising applying a formulation according to the first aspect of the present invention to the wound of a subject.

In any method of the third aspect of the present invention, the subject may be a human or non-human mammal. Preferably the subject is a human.

A fourth aspect of the present invention provides a method of preparing a formulation according to the first aspect of the present invention, the method comprising the steps of:
(a) dissolving the pharmaceutically active agent and, if present, the acid or base in water;
(b) adding the polyethylene glycol or poloxamer, the polyethylene glycol mono- or di-ether and, if present, the alcohol, the penetration enhancer and the plasticizer to the solution; and
(c) stirring the mixture until a hydrophilic gel is obtained.

In a preferred embodiment, the pharmaceutically active agent can be protonated and an acid is used in step (a), which protonates the pharmaceutically active agent. A preferred pharmaceutically active agent, which can be protonated, is abafungin or a pharmaceutically acceptable salt thereof. In an alternative embodiment, the pharmaceutically active agent can be deprotonated and a base is used in step (a), which deprotonates the pharmaceutically active agent.

For the avoidance of doubt, insofar as is practicable any embodiment of a given aspect of the present invention may occur in combination with any other embodiment of the same aspect of the present invention. In addition, insofar as is practicable it is to be understood that any preferred or optional embodiment of any aspect of the present invention should also be considered as a preferred or optional embodiment of any other aspect of the present invention.

In addition, it is also to be understood that any lower limit specified in connection with a variable of the preset invention may be combined with any upper limit specified in connection with the same variable so as to form a range that is also encompassed by the present invention.

### BRIEF DESCRIPTION OF THE DRAWINGS

FIG. 1 shows three horse hoof horn membranes (labelled 1, 2 and 3) 24 hours after the application of three formulations comprising abafungin.

FIG. 2 is a graph showing the amount of abafungin which has penetrated into horse hoof horn membranes 24 hours after the application of three formulations comprising abafungin.

FIG. 3 shows the toenail of a volunteer suffering from onychomycosis after topical application of a formulation of the present invention comprising abafungin.

FIG. 4 shows the toenails of another volunteer suffering from onychomycosis before treatment and after oral itraconazole administration and concurrent topical application of a formulation of the present invention comprising abafungin. In

FIG. 4, ‘(1)’ refers to the oral itraconazole administration, and ‘Abagel 10%’ refers to the topical application of the abafungin formulation.

FIG. 5 is a graph showing the amount of abafungin, ciclopirox or ciclopirox olamine which has penetrated into horse hoof horn membranes 24 hours after the application of five formulations comprising abafungin, ciclopirox or ciclopirox olamine.

FIG. 6 is a graph showing the amount of abafungin or hydrocortisone which has penetrated into porcine ear skin 24 hours after the application of four formulations comprising abafungin or hydrocortisone.

FIG. 7 is a graph showing the percentage deviation of TEWL (transepidermal water loss) measurements one hour after treatment with Batrafen®, Loceryl® or a formulation according to the present invention to the measurements before the treatment.

FIG. 8 shows a schematic diagram of a fungal inhibition zone.

FIG. 9 are photographs of Sabouraud plates inoculated with T. rubrum 34 and treated with bovine hoof horn membrane treated with four formulations comprising abafungin.

### DETAILED DESCRIPTION OF THE INVENTION

A preferred formulation of the present invention is a water-based, hydrophilic, non-irritating gel formulation suitable for the treatment of onychomycosis, dermatomycosis and other mycoses (see examples 5 and 10). The formulation comprises a polyethylene glycol mono- or di-ether (preferably polyethylene glycol monomethyl ether (MPEG)) and a polyethylene glycol or a poloxamer (preferably polyethylene glycol (PEG)) as adhesives and film formers, which ensure that the formulation is capable of releasing pharmaceutically active agents slowly.

Polyethylene glycols and poloxamers, in particular PEG, are known permeation enhancers and known for the sustained release of pharmaceutically active agents. Polyethylene glycol ethers, in particular MPEG, are solubilisers and film builders. Without wishing to be bound by theory, it is thought that together they act as adhesives and film formers and ensure that the formulation of the present invention forms a breathable film incorporating a pharmaceutically active agent. The water naturally present in the nail or skin dissolves the pharmaceutically active agent out of the PEG or poloxamer/PEG-ether depot, which releases the pharmaceutically active agent slowly. The presence of the polyethylene glycol ether is thought to lead to higher interactions of all substances concerned, for example, the ether group is thought to lead to greater adhesion of the formulation to the organic nail or skin material. The ether group is also thought to be responsible for the observed high solubility of lipophilic and/or keratinophilic pharmaceutically active agents in the formulation.

Known water-based formulations use swelling gel builders (e.g. hydroxymethyl cellulose) and/or water-soluble acrylic acid copolymers. These gel builders can be used in a concentration of only up to 1.5% in water, since otherwise the viscosity of the formulation becomes too high. However, such small amounts of gel builders are not enough to provide an effective depot for a pharmaceutically active agent.

The hydrophilic gel formulations according to the present invention, on the other hand, act as a depot for the pharmaceutically active agent. The water naturally present in the nail or skin dissolves the pharmaceutically active agent out of the PEG or poloxamer/PEG-ether depot, which releases the pharmaceutically active agent slowly, providing for a modified release such as delayed, extended, sustained or controlled release.

Pharmaceutically active agents suitable for use in the formulation of the present invention include lipophilic and/or keratinophilic substances, e.g. anti-mycotics, which can be applied to the nail, skin and mucosa for the treatment of onychomycosis, dermatomycosis and other mycoses, such as oral, vaginal and rectal mycoses. Suitable anti-mycotics include, but are not limited to azoles (such as imidazoles and triazoles), imidazoles (such as bifonazole, clotrimazole, econazole, fenticonazole, isoconazole, ketoconazole, miconazole, oxiconazole, tioconazole, sertaconazole, and sulconazole), triazoles (such as fluconazole, itraconazole, posaconazole, ravuconazole, terconazole, and voriconazole), thiazoles (such as 2-amino-thiazoles such as abafungin), thiadiazoles, guanidines (such as arylguanidines such as abafungin), pyrimidines (such as pyrimidinimines such as abafungin), imines (such as pyrimidinimines such as abafungin), morpholines (such as amorolfine), 2-pyridones (such as ciclopirox), 2-pyrimidones (such as flucytosine), allylamines (such as terbinafine and naftifine), benzylamines (such as butenafine), polyenes (such as amphotericin B, nystatin, and pimaricin (also called natamycin)), echinocandins (such as caspofungin, micafungin, and anidulafungin), benzofurans, benzoxaboroles, pyridines, thiocarbamates, and others.

In a preferred embodiment of the present invention, the formulation comprises abafungin, ciclopirox olamine, terbinafine hydrochloride, or amorolfine; more preferably abafungin. A formulation comprising abafungin also preferably comprises an acid for adjusting the pH of the formulation, such that the abafungin in the formulation is protonated into the active molecule, the guanidinium ion.

Lipophilic and/or keratinophilic pharmaceutically active agents, such as abafungin, ciclopirox olamine and terbinafine hydrochloride, are surprisingly stable and soluble in the formulation of the present invention. For example, abafungin is lipophilic and poorly soluble in many excipients (see example 1). It is therefore very difficult to solubilise a pharmaceutically active agent such as abafungin in a pharmaceutical formulation in an amount sufficient for an acceptable permeation rate (see examples 2, 3, 4, 6 and 7). The PEG or poloxamer/PEG-ether mixture of the formulation of the present invention makes it possible for a lipophilic and/or keratinophilic pharmaceutically active agent such as abafungin to be solubilised adequately and the PEG or poloxamer/PEG-ether mixture is thought to prevent the lipophilic and/or keratinophilic pharmaceutically active agent from crystallising out of the formulation. For example, a concentration of up to 30% abafungin can be achieved in the formulation of the present invention (see example 8).

Pharmaceutically active agents, such as abafungin, also showed surprisingly much higher permeation rates into and across the nail and into the skin from the formulation of the present invention compared to conventional lacquer formulations and compared to hydrophilic nail gels without a polyethylene glycol ether (see examples 2, 3, 4, 6 and 7).

Human nails behave like hydrophilic membranes and have a high transungual diffusion of water (1.8 to 3.1 mg/cm²) (K. A. Walters et al., Journal of Pharmacy and Pharmacology, 1985, vol. 37, pages 771-775; D. Mertin et al., Journal of Pharmacy and Pharmacology, 1997, vol. 49, pages 30-34; Y. Kobayashi et al., European Journal of Pharmaceutical Sciences, 2004, vol. 21, pages 471-477). The permeability of the nail to water is some 1000-fold greater than that of the stratum corneum (D. Spruit, Journal of Investigative Dermatology, 1971, vol. 56, pages 359-361; K. A. Walters et al., Journal of Investigative Dermatology, 1981, vol. 36, pages 101-103).

For a healthy nail, a high transungual diffusion of free nail water is of great importance. The formulations according to the present invention are hydrophilic water-based gels, which allow water to pass into and out of the nail after application of the formulations to the nail. This is in contrast to conventional lacquers, which reduce the transungual diffusion of water significantly (de Berker & Baran, Int. J. Cosmetic Science, 2007, vol. 29, pages 241-275; Spruit, Am. Cosmet. Perfum., 1972, vol. 87, pages 57-58). This was also confirmed by example 9 below.

It is currently believed that because water is able to permeate freely across the nail and into the formulation according to the present invention, the pharmaceutically active agent contained in the formulation will be dissolved over time out of the gel formulation into the nail. Conventional lacquers, using water insoluble polymers as film builders, cover the nail and inhibit the free permeation of nail water, and thus the dissolution of a pharmaceutically active agent out of the hydrophobic lacquer film is much lower compared to a hydrophilic gel formulation according to the present invention.

Conventional anti-mycotic nail lacquers, such as Penlac® (also called Batrafen®) (ciclopirox) from Aventis and Loceryl® (amorolfine) from Galderma, use alcohols as solvents and water insoluble polymers. Therefore the lacquer films formed from such conventional lacquers are water insoluble and the water which is naturally present in nails cannot dissolve the anti-mycotic agents out of the water insoluble polymer matrices. This results in a slow penetration rate of the anti-mycotic agents from the conventional lacquers into the nail. The formulations of the present invention on the other hand are hydrophilic and the pharmaceutically active agents move easily from the hydrophilic formulations into the nail water.

Moreover, conventional nail lacquers irritate and damage the skin and therefore cannot be used on skin. The formulation of the present invention on the other hand allows pharmaceutically active agents to penetrate through the skin surrounding the nail into the nail bed and nail matrix.

The formulation of the present invention can deliver an anti-fungal or anti-mycotic agent to the nail plate (the stratum corneum unguis) and to the nail bed (the modified area of the epidermis beneath the nail, over which the nail plate slides as it grows) through the nail plate and around the nail periphery. Desirably the anti-fungal or anti-mycotic agent is also concurrently delivered to the nail matrix, the cuticle and the hyponychium (the thickened epidermis underneath the free distal end of a nail).

The hydrophilic nature of the formulation of the present invention simulates the conditions and characteristics of a human nail, especially the hydrophilic membranes of the nail. Polyethylene glycols and poloxamers have an occlusive effect which enhances the level of hydration of the nail. Moreover, unlike conventional nail lacquers, the PEG or poloxamer/PEG-ether mixture of the formulation of the present invention is skin compatible and breathable. Preferably the formulation has a pH of about 5.5, which simulates the conditions of human skin. When applied to the nail of a patient, the formulation of the present invention is thought to allow a pharmaceutically active agent to penetrate into the patient's nail and nail bed including the nail matrix in two ways, namely through the nail itself and through the skin surrounding the nail. Therefore another advantage of the formulations of the present invention is the two-way transport of the pharmaceutically active agent into and across the hydrophilic nail: transungual and transdermal. The onychomycosis will be treated by an application of the formulations of the present invention not only on the nail, but also on the surrounding skin area.

Another advantage of the formulation of the present invention is that compared to other hydrophilic gels (e.g. on the basis of hydroxymethyl cellulose or PEG), the PEG or poloxamer/PEG-ether mixture showed surprisingly excellent drying times that are comparable or even better than those of conventional lacquers (e.g. based on polyvinylacetate, (meth)acrylic acid alkyl ester copolymers, or methylvinyl ether maleic acid monoalkyl ester copolymers).

Moreover, unlike conventional nail lacquers, the formulation of the present invention can be washed off. This results in better patient compliance, because it avoids the need for time consuming removal of conventional nail lacquers by filing or the use of solvent based formulations. Standard nail lacquers have to be removed at least weekly with alcoholic wipes and by using a nail file. Especially for older patients, this therapy plan is difficult to adopt. Moreover, the use of a nail file can induce severe injuries of the skin surrounding the nail, which can result in systemic uptake of fungi. The formulations of the present invention will ease the therapy plan for patients, because the formulations can be removed easily by washing. Therefore the formulations of the present invention increase patient compliance.

### Examples

### Example 1

### Abafungin Solubility

To order to study the solubility of abafungin, abafungin was dissolved in a number of excipients. The results of the solubility studies are summarised in Table 1.

TABLE 1

| Excipient group | Excipient | Soluble | Not soluble |
|---|---|---|---|
| cosmetic oils | isopropyl palmitate | | ✓ |
| | isopropyl myristate | | ✓ |
| | cetearyl ethylhexanoate | | ✓ |
| | decyl oleate | | ✓ |
| | medium chain triglyceride | | ✓ |
| | transcutol | ✓ (3%) | |
| water | water | | ✓ |
| monohydric | ethanol | | ✓ |
| alcohols | ethanol 70% | | ✓ |
| | isopropanol | | ✓ |
| polyhydric alcohols | propylene glycol | | ✓ |
| | glycerine | | ✓ |
| polyethylene glycols | PEG 20000 | | ✓ |
| | PEG 12000 | | ✓ |
| | PEG 6000 | | ✓ |
| | PEG 4500 | | ✓ |
| | PEG 1500 | | ✓ |
| | PEG 400 | | ✓ |
| polyethylene glycol | MPEG 2000 | | ✓ |
| monomethyl ethers | MPEG 550 | | ✓ |

Abafungin is insoluble in most excipients, even in each of water, polyethylene glycol and polyethylene glycol monomethyl ether. However, surprisingly, it was found that abafungin is soluble in a mixture of water, polyethylene glycol, polyethylene glycol monomethyl ether and an acid such as formic acid.

### Example 2

### Proximal Flux and Affinity of Three Abafungin Formulations into Horse Hoof Horn Membranes

In order to study the ability of abafungin to penetrate into nails, three abafungin formulations were prepared, comprising the ingredients set out in Table 2. Formulations 1 and 2 were hydrophilic gels, and formulation 3 was a lacquer. Formulation 2 is according to the present invention, and formulations 1 and 3 are comparative formulations.

TABLE 2

| | Formulation 1 | Formulation 2 | Formulation 3 |
|---|---|---|---|
| | amounts (%) | amounts (%) | amounts (%) |
| Abafungin | 10 | 10 | 10 |
| 2-Propanol | 37 | 37 | — |
| PEG 20000 | 18.4 | 18.4 | — |
| PEG 8000 | 3 | 3 | — |
| MPEG 2000 | — | 5 | — |
| Water | 24 | 20 | — |
| Formic acid | 1.6 | 1.6 | — |
| Isopropyl myristate | 0.5 | 0.5 | — |
| Transcutol | 3.5 | 3.5 | — |
| Propylene glycol | 1 | 1 | — |
| Hydroxyethyl cellulose | 1 | — | — |
| Gantrez ES 425 | — | — | 30 |
| Ethyl acetate | — | — | 17.2 |
| Butyl acetate | — | — | 5.7 |
| Triacetin | — | — | 1.2 |
| Miglyol 812N | — | — | ad. 100 ml |

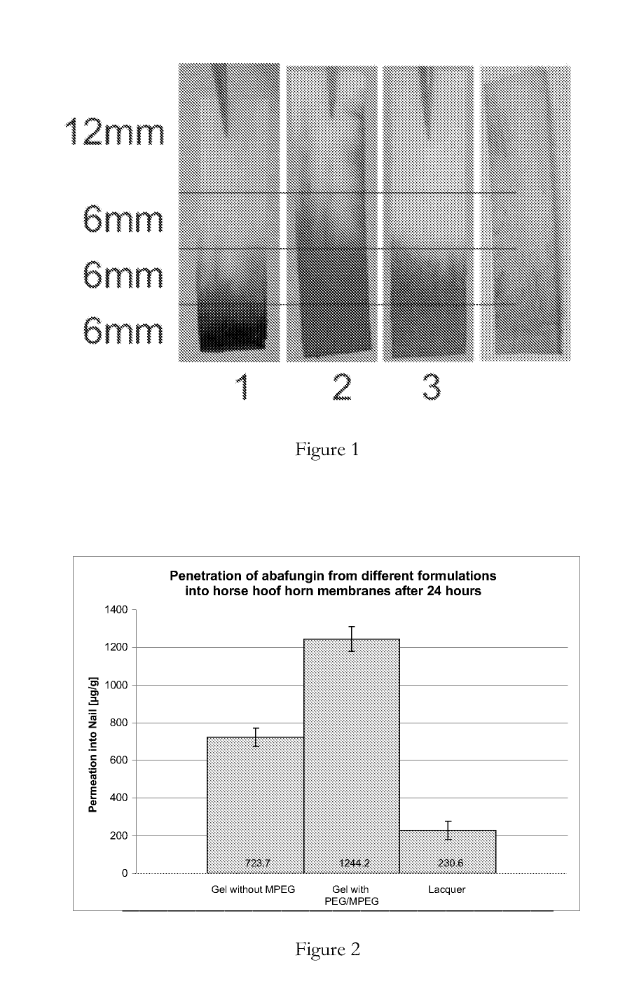

The formulations were applied to horse hoof horn membranes of about 600-700 μm thickness for 24 hours to ascertain the amount of abafungin penetration. The horse hoof horn membranes are shown in FIG. 1 and the results are summarised in Table 3.

TABLE 3

| | μg/g abafungin in horse hoof horn membranes | | |
|---|---|---|---|
| | (+/−S.D., n = 3, after 24 h) | | |
| [mm] | Formulation 1 | Formulation 2 | Formulation 3 |
| 0-6 | 2332.47 +/− 654.56 | 2546.14 +/− 856.13 | 2532.59 +/− 757.66 |
| 6-12 | 1980.54 +/− 612.45 | 2617.71 +/− 944.71 | 2157.89 +/− 916.72 |
| 12-18 | 274.23 +/− 139.26 | 2618.28 +/− 903.49 | 488.44 +/− 388.52 |
| 18-30 | 53.87 +/− 47.68 | 1559.44 +/− 461.42 | 105.57 +/− 97.29 |
| Total | 4641.11 | 9341.57 | 5284.49 |

When applied in the formulation according to the present invention (formulation 2), abafungin penetrated the horse hoof horn membranes much better, namely more in total (9341.57 μg/g compared to 4641.11 μg/g and 5284.49 μg/g) and further in distance (higher proportion in the 18-30 mm penetration distance), than when applied in the comparative formulations (formulations 1 and 3).

### Example 3

### Ex vivo Penetration Studies of Three Abafungin Formulations into Horse Hoof Horn Membranes

In order to simulate human in vivo conditions, ex vivo penetration studies on horse hoof horn membranes were performed. Animal hoof is made of essentially the same material as human nails. Horse hoof was sawn into horn membranes having an area of about 2 cm² and a thickness of 600-700 μm which conforms to human nails. Human finger nails are about 500 μm thick and human toenails about 800 μm.

1 ml of each of formulations 1, 2 and 3 of example 2 was applied to a horse hoof horn membrane. The horse hoof horn membranes were placed in Franz diffusion cells (area 1.76 cm²) and the cells were filled with a tempered blood simulating buffer (phosphate buffered saline). The buffer was stirred at 300 rpm. After 24 hours, the horse hoof horn membranes were removed from the Franz diffusion cells and residues of the formulations were removed. The effective penetration area of 1.76 cm² was cut into small pieces and abafungin was extracted using a mixture of 80% acetonitrile, 19.6% water and 0.4% perchloric acid. The samples were extracted for 30 minutes using an ultrasonic water bath at 60° C. The supernatant was analysed using HPLC.

The results are presented in FIG. 2. When applied in the formulation according to the present invention (formulation 2), more abafungin penetrated the horse hoof horn membranes than when applied in the comparative formulations (formulations 1 and 3).

### Example 4

### Penetration of Abafungin into Stratum Corneum and Epidermis/Dermis

In order to study the ability of abafungin to penetrate into skin, penetration studies with the abafungin formulation 2 of example 2 were performed. Penetration tests with unstripped porcine ear skin (thickness 2 mm) were performed using Franz diffusion cells (buffer conditions: thermo jacket 36° C., 300 rpm, BPS buffer). 1 ml of formulation 2 of example 2 was applied onto the skin. After 24 hours incubation, the stratum corneum was removed and abafungin was extracted from both the stratum corneum and the epidermis/dermis, with 1 ml of a mixture of 80% acetonitrile, 19.6% water and 0.4% perchloric acid at 60° C. for 1.5 hours. The supernatant was analysed using HPLC. It was found that fungicidal concentrations (16-30 μg/ml) of abafungin had been achieved in both the stratum corneum and the epidermis/dermis (Franz diffusion cells, n=3). The abafungin concentration in the epidermis/dermis was found to be 32.19±1.19 μg/g, and in the stratum corneum 4617.50±731.86 μg/g.

### Example 5

### Abafungin for the Treatment of Onychomycosis

A hydrophilic gel formulation according to the present invention was prepared, comprising the ingredients set out in Table 4. The formulation was the same as formulation 2 in example 2.

TABLE 4

| Ingredient | Amount (%) |
|---|---|
| 2-Propanol | 37.0 |
| Abafungin | 10.0 |
| Water | 20.0 |
| Formic acid | 1.6 |
| PEG 20000 | 18.4 |
| PEG 8000 | 3.0 |
| MPEG 2000 | 5.0 |
| Isopropyl myristate | 0.5 |
| Transcutol | 3.5 |
| Propylene glycol | 1.0 |
| Total | 100 |

The gel formulation was prepared by dissolving abafungin and formic acid in water. Then the remaining ingredients (namely 2-propanol, PEG 20000, PEG 8000, MPEG 2000, isopropyl myristate, transcutol, and propylene glycol) were added to this solution and the mixture was stirred until a gel formulation was formed.

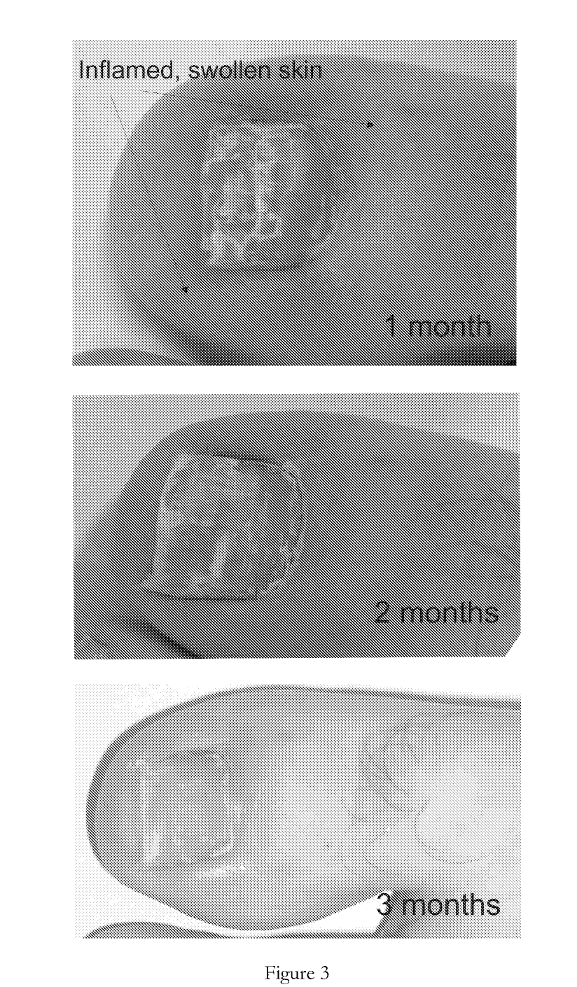

The gel formulation was applied once daily to the left toenail of a male volunteer (aged 32) suffering from onychomycosis. The results are shown in FIG. 3, which shows the toenail (a) after one month, (b) after two months, and (c) after three months of once daily application of the formulation. There is a marked improvement in the toenail's condition.

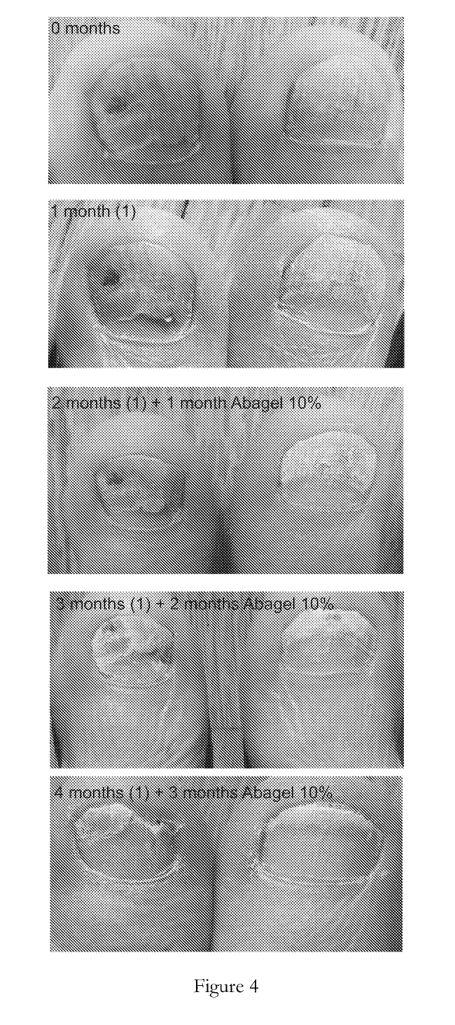

A second male volunteer (aged 55), also suffering from onychomycosis, was treated orally with 100 mg itraconazole (Itracol®) twice daily for one week followed by three weeks intermission. After one month of itraconazole administration, the volunteer additionally applied the gel formulation once daily to his toenails. The results are shown in FIG. 4, which shows the toenails (a) before treatment, (b) after one month of itraconazole administration, (c) after two months of itraconazole administration and one month application of the abafungin formulation, (d) after three months of itraconazole administration and two months application of the abafungin formulation, and (e) after four months of itraconazole administration and three months application of the abafungin formulation. There is a marked improvement in the toenails' condition.

### Example 6

### Ex vivo Penetration Studies of Five Formulations Comprising Abafungin, Ciclopirox or Ciclopirox Olamine into Horse Hoof Horn Membranes

In order to simulate human in vivo conditions, ex vivo penetration studies on horse hoof horn membranes were performed. Animal hoof is made of essentially the same material as human nails. Horse hoof was sawn into horn membranes having an area of about 2 cm² and a thickness of 600-700 μm which conforms to human nails. Human finger nails are about 500 μm thick and human toenails about 800 μm.

Formulations 1-3 and 5 were prepared and formulation 4 was purchased, comprising the ingredients set out in Table 5. Formulations 1, 2 and 5 were hydrophilic gels, and formulations 3 and 4 were lacquers. Formulations 2 and 5 are according to the present invention, and formulations 1, 3 and 4 are comparative formulations.

TABLE 5

| | Formulation 1 | Formulation 2 | Formulation 3 | Formulation 4* | Formulation 5 |
|---|---|---|---|---|---|
| | amounts (%) | amounts (%) | amounts (%) | amounts (%) | amounts (%) |
| Abafungin | 10 | 10 | 10 | — | — |
| Ciclopirox | — | — | — | 8 | — |
| Ciclopirox olamine | — | — | — | — | 8 |
| 2-Propanol | 37 | 37 | — | yes | 37 |
| PEG 20000 | 18.4 | 18.4 | — | — | 18.4 |
| PEG 8000 | 3 | 3 | — | — | 3 |
| MPEG 2000 | — | 5 | — | — | 5 |
| Water | 24 | 20 | — | — | 22 |
| Formic add | 1.6 | 1.6 | — | — | 1.6 |
| Isopropyl myristate | 0.5 | 0.5 | — | — | 0.5 |
| Transcutol | 3.5 | 3.5 | — | — | 3.5 |
| Propylene glycol | 1 | 1 | — | — | 1 |
| Hydroxyethyl cellulose | 1 | — | — | — | — |
| Gantrez ES 425 | — | — | 30 | — | — |
| Ethyl acetate | — | — | 17.2 | yes | — |
| Butyl acetate | — | — | 5.7 | — | — |
| Triacetin | — | — | 1.2 | — | — |
| Miglyol 812N | — | — | ad. 100 ml | — | — |
| Poly(butylhydrogen- | — | — | — | yes | — |
| maleate, methoxy- | | | | | |
| ethylene) (1:1) | | | | | |

*commercially available Batrafen ® (also called Penlac ®), therefore amounts of excipients unknown

250 μl of each of formulations 1-5 was applied to a horse hoof horn membrane. The horse hoof horn membranes were placed in Franz diffusion cells (area 1.76 cm²) and the cells were filled with a tempered blood simulating buffer (phosphate buffered saline). The buffer was stirred at 300 rpm. After 24 hours, the horse hoof horn membranes were removed from the Franz diffusion cells and the residues of the formulations were removed. The effective penetration area of 1.76 cm² was cut into small pieces and the API (abafungin, ciclopirox, or ciclopirox olamine) was extracted using an appropriate solvent. The samples were extracted for 30 minutes using an ultrasonic bath at 60° C. The supernatant was analysed using HPLC.

TABLE 6

| | | [mg/g nail] +/− | [mg/cm² nail] +/− | number of |
|---|---|---|---|---|
| Formulation | API | S.D. | S.D. | cells |
| 1 | 10% abafungin | 0.71000 +/− 0.05000 | 0.05244 +/− 0.01468 | 3 |
| 2 | 10% abafungin | 1.96571 +/− 0.50360 | 0.14432 +/− 0.04222 | 7 |
| 3 | 10% abafungin | 0.47377 +/− 0.20475 | 0.03377 +/− 0.01374 | 7 |
| 4 | 8% ciclopirox | 1.20527 +/− 0.35257 | 0.06639 +/− 0.01802 | 7 |
| 5 | 8% ciclopirox | 2.39251 +/− 0.07734 | 0.18347 +/− 0.00642 | 3 |
| | olamine | | | |

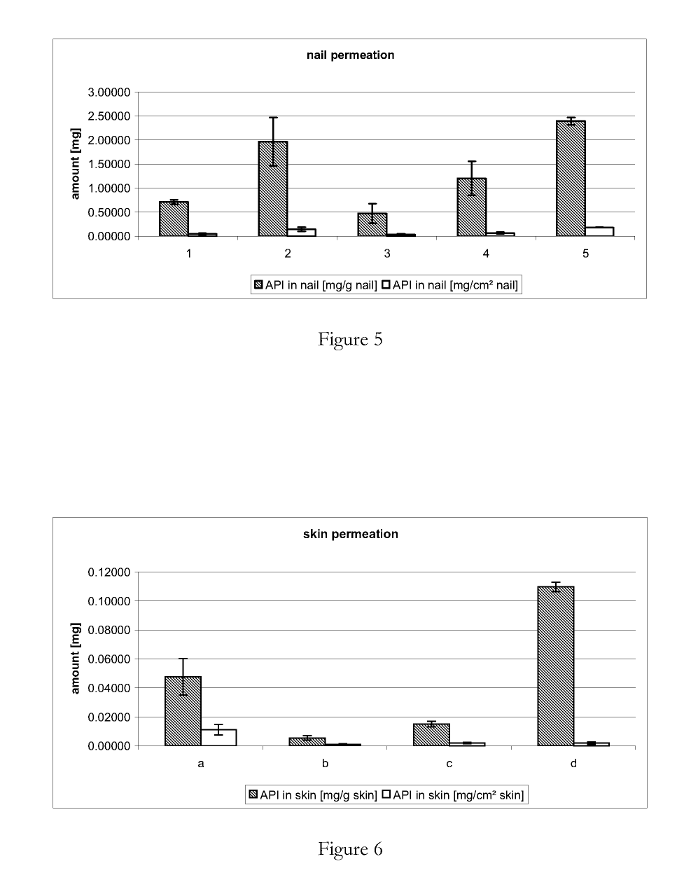

The results are presented in Table 6 and FIG. 5. When applied in the formulations according to the present invention (formulations 2 and 5), more abafungin and ciclopirox olamine penetrated the horse hoof horn membranes than when applied in the comparative formulations (formulations 1, 3 and 4).

The in vitro trials with ciclopirox in a formulation according to the present invention (formulation 5) demonstrate higher penetration rates into the nail in comparison to the marketed ciclopirox lacquer used in Batrafen® (formulation 4).

### Example 7

### Ex vivo Penetration Studies of Four Formulations Comprising Abafungin or Hydrocortisone into Porcine Ear Skin

In order to simulate human in vivo conditions, ex vivo penetration studies on porcine ear skin were performed. Porcine ear skin is made of essentially the same material as human skin. Porcine ear skin was removed carefully from the chondral tissue and cut into pieces having an area of about 2 cm² and a thickness of about 2000 μm which conforms to human skin.

Formulations a, b and d were prepared and formulation c was purchased, comprising the ingredients set out in Table 7. Formulations a and d were hydrophilic gels according to the present invention, and formulations b and c were comparative cream formulations.

TABLE 7

| | | | Formu- | Formu- |
|---|---|---|---|---|
| | Formulation | Formulation | lation c* | lation d |
| | a | b | amounts | amounts |
| | amounts (%) | amounts (%) | (%) | (%) |
| Abafungin | 1 | 1 | — | — |
| Hydrocortisone | — | — | 1 | 1 |
| 2-Propanol | 29 | — | — | 29 |
| PEG 400 | 8 | — | — | 8 |
| PEG 8000 | 21.4 | — | — | 21.4 |
| MPEG 2000 | 5 | — | — | 5 |
| Water | 24 | yes | yes | 24 |
| Formic acid | 0.6 | — | — | 0.6 |
| Isopropyl myristate | 0.5 | — | yes | 0.5 |
| Transcutol | 3.5 | — | — | 3.5 |
| Propylene glycol | 7 | — | — | 7 |
| 2-Octyldodecanol | — | yes | — | — |
| Cetostearyl alcohol | — | yes | — | — |
| Cetyl palmitate | — | yes | — | — |
| Polysorbate 60 | — | yes | — | — |
| Sorbitan monostearate | — | yes | — | — |
| Stearic acid | — | yes | — | — |
| Benzyl alcohol | — | yes | — | — |
| Urea | — | — | yes | — |
| White vaseline | — | — | yes | — |
| Maize starch | — | — | yes | — |
| Sorbitan laurate | — | — | yes | — |
| Sorbitol solution | — | — | yes | — |
| Poly(oxyethylene)-25 | — | — | yes | — |
| hydrogenated | | | | |
| castor oil | | | | |

*commercially available Hydrodexan Crème ®, therefore amounts of excipients unknown

250 μl of each of formulations a-d was applied to a porcine ear skin piece. The porcine ear skin pieces were placed in Franz diffusion cells (area 1.76 cm²) and the cells were filled with a tempered blood simulating buffer (phosphate buffered saline). The buffer was stirred at 300 rpm. After 24 hours, the skin pieces were removed from the Franz diffusion cells and the residues of the formulations were removed. The effective penetration area of 1.76 cm² was cut into small pieces and the API (abafungin or hydrocortisone) was extracted using an appropriate solvent. The samples were extracted for 30 minutes using an ultrasonic bath at 60° C. The supernatant was analysed using HPLC.

TABLE 8

| | | [mg/g skin] +/− | [mg/cm² skin] +/− | number of |
|---|---|---|---|---|
| Formulation | API | S.D. | S.D. | cells |
| a | 1% abafungin | 0.04751 +/− 0.01262 | 0.01110 +/− 0.00368 | 6 |
| b | 1% abafungin | 0.00535 +/− 0.00165 | 0.00096 +/− 0.00036 | 6 |
| c | 1% hydrocortisone | 0.01500 +/− 0.00190 | 0.00183 +/− 0.00042 | 5 |
| d | 1% hydrocortisone | 0.10960 +/− 0.00328 | 0.00185 +/− 0.00085 | 5 |

The results are presented in Table 8 and FIG. 6. When applied in the formulations according to the present invention (formulations a and d), more abafungin and hydrocortisone penetrated into porcine ear skin than when applied in the comparative formulations (formulations b and c).

The hydrocortisone formulation according to the present invention (formulation d) enhances the penetration rate of the cortico steroid hydrocortisone in comparison to the marketed formulation Hydrodexan Creme® (formulation c).

### Example 8

### Solubility and Stability Studies with Abafungin, Hydrocortisone and Ciclopirox Olamine

The solubility and stability of abafungin, hydrocortisone and ciclopirox olamine were tested in a formulation according to the present invention, in a standard ethanol gel (Ethanolhaltiges Erythromycin Gel, NRF 11.84, ABDA, Govi Verlag Pharmazeutischer Verlag GmbH, Eschborn), in pure water and in pure ethanol. The maximum solubilities of the three APIs without crystallisation in the different formulations are summarised in Table 9.

TABLE 9

| | | | Ciclopirox |
|---|---|---|---|
| | Abafungin | Hydrocortisone | Olamine |
| Formulation according to | ~30%*² | ~2.7% | ~11.0% |
| the present invention*¹ | | | |
| Formulation according | ~102.6% | ~103.7% | ~104.4% |
| to the present | ([c] 10%)*⁴ | ([c] 1%)*⁴ | ([c] 8%)*⁴ |
| invention*³ after 3 | | | |
| months at 24° C. | | | |
| (HPLC recovery) | | | |
| Ethanol gel (NRF 11.84) | 0.2% | ~1.2% | ~soluble*⁵ |
| Pure water | <0.00002% | ~0.028% | ~1-3%*⁵ |
| Pure ethanol | <0.24% | ~1.5% | ~soluble*⁵ |

*¹formulation comprising the same excipients in the same ratios as formulation 2 of example 2/6, with abafungin, hydrocortisone or ciclopirox olamine being added gradually
*²dependent on pH
*³same as formulation 2 of example 2/6, formulation d of example 7, and formulation 5 of example 6 respectively
*⁴initial API concentration
*⁵not measured, data according to Neues Rezeptur-Formularium ABDA, Bundesvereinigung Deutscher Apothekerverbände, Pharmazeutisches Laboratorium, Govi Verlag Pharmazeutischer Verlag GmbH, Eschborn

It was found that the formulation according to the present invention prevents crystallisation and increases the solubility and stability of the three APIs (abafungin, hydrocortisone and ciclopirox olamine) in comparison to the standard ethanol gel, pure water and pure ethanol.

### Example 9

### TEWL (Transepidermal Water Loss) Studies

The influence of the application of standard nail lacquers (Batrafen® and Loceryl®) and of a formulation according to the present invention on TEWL (transepidermal water loss) was studied.

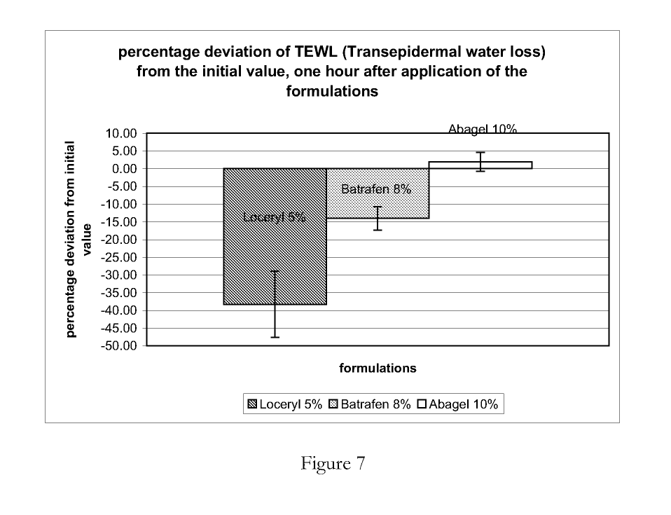

The thumbnails of three volunteers were treated with three different formulations, namely a formulation according to the present invention (formulation 2 of example 2) and commercially available lacquer formulations Batrafen® and Loceryl®. The TEWL of the thumbnail was measured before and one hour after treatment of the three volunteers. The results are presented in FIG. 7, which shows the percentage deviation of the measurements one hour after treatment to the measurements before the treatment.

Both, the Batrafen® and Loceryl® lacquers resulted in a significant reduction of water loss and humidity above the nail. Only with the formulation according to the present invention, the free nail water can still permeate freely across the nail plate, moisten the nail plate and dissolve the pharmaceutically active agent out of the hydrophilic gel formulation into the nail.

### Example 10

### Fungal Inhibition Assays

The objective of this study was to determine the ability of abafungin, formulated according to the present invention, to permeate through bovine hoof horn membrane (a model of human nail) and inhibit the growth of Trichophyton rubrum 34. T. rubrum is the most prevalent pathogen responsible for onychomycosis of the toenail (W. K. Foster, M. A. Ghannoum and B. E. Elewski, J. Am. Acad. Dermatol., 2004, vol. 50, pages 748-752). One formulation according to the present invention (A) was tested alongside three alternative abafungin formulations not according to the present invention (B-D) for comparative purposes.

Bovine hoof horn membranes were hydrated in sterile distilled water in petri dishes for 2 hours. Subsequently, the bovine hoof horn membranes were removed from the petri dishes and dried on a filter paper.

To prepare an inoculum of T. rubrum 34, 1-2 ml of sterile saline was added to the surface of the corresponding colony in agar gel in petri dishes and the surface was agitated with a swab. The suspension was then transferred to a Universal tube and its turbidity was adjusted (using sterile saline or suspension) to a McFarland Standard 2. The surfaces of fresh Sabouraud agar plates were swabbed using the inoculum.

Drug formulations A-D were applied onto the bovine hoof horn membranes. A blank, untreated bovine hoof horn membrane was used as a control. Once the treatments had dried, the bovine hoof horn membranes were placed with the treated surface uppermost in the middle of an inoculated Sabouraud plate and the plates were incubated at 27° C. for 5 days. Three repetitions of each test condition were performed.

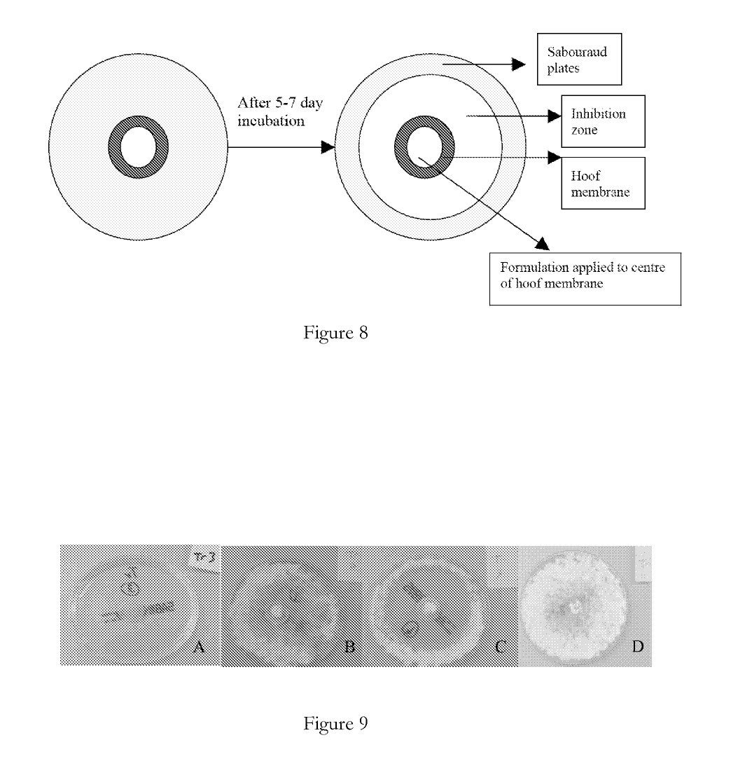

Following incubation for 5 days, an inhibition zone was observed (see FIG. 8), as the drug permeated into the bovine hoof horn membrane and through the latter into the agar gel. Photographs were taken of the agar plates (see FIG. 9) and the diameter of the inhibition zone was calculated as per the following equation:

$$Diameter ( dia ) of inhibition zone ( mm ) = dia of inhibition zone on photograph \times \frac{real dia of petri dish}{dia of petri dish on photograph}$$

The results are presented in Table 10, which summarises the diameter of the inhibition zones of T. rubrum 34 following incubation of the bovine hoof horn membranes treated with formulations A-D. The results of the three repetitions of each test condition are provided.

TABLE 10

| | | | Diameter of inhibition zone (mm) | | | |
|---|---|---|---|---|---|---|
| | | Abafungin | | | | Ave ± |
| Formulation | | concentration | 1 | 2 | 3 | S.D. |
| — | Blank bovine hoof horn membrane | — | 0 | 0 | 0 | 0 |
| | (photograph not shown) | | | | | |
| A | Formulation according to the present | 10% | 88 | 88 | 88 | 88 ± 0 |
| | invention** | | | | | |
| B | Lacquer, not according to the present | 10.7% | 31* | 39 | 27* | — |
| | invention | | | | | |
| C | Gel 1, not according to the present | 10% | 51 | 57 | 62 | 57 ± 5.5 |
| | invention | | | | | |
| D | Gel 2, not according to the present | 5.1% | 40* | 28 | 33* | — |
| | invention | | | | | |

*agar gel within inhibition zone did not become completely clear, but the density of fungi within inhibition zone was less than outside of inhibition zone
**same as formulation 2 of example 2

The application of the abafungin formulation according to the present invention (A) resulted in complete clearance of the plate. This was not achieved by application of any of the three alternative abafungin formulations not according to the present invention (B-D).

1. A formulation comprising: (a) a pharmaceutically active agent; (b) water;
(c) a polyethylene glycol (PEG) or a poloxamer; and (d) a polyethylene glycol mono- or di-ether.

2. The formulation of claim 1, wherein the mean molecular weight of the polyethylene glycol is in the range of 200-100000.

3. The formulation of claim 1, wherein the polyethylene glycol is PEG 8000-20000.

4. The formulation of claim 1, wherein the mean molecular weight of the poloxamer is in the range of 1000-16000.

5. The formulation of claim 1, wherein the formulation comprises the polyethylene glycol or poloxamer in an amount of 5-50%.

6. The formulation of claim 1, wherein the formulation comprises a polyethylene glycol.

7. The formulation of claim 1, wherein the polyethylene glycol mono- or di-ether is an alkyl, aryl, arylalkyl or alkylaryl ether.

8. The formulation of claim 1, wherein the polyethylene glycol mono- or di-ether is an alkyl ether.

9. The formulation of claim 1, wherein the polyethylene glycol mono- or di-ether is a methyl or ethyl ether.

10. The formulation of claim 1, wherein the polyethylene glycol mono- or di-ether is a mono-ether.

11. The formulation of claim 1, wherein the mean molecular weight of the polyethylene glycol mono- or di-ether is in the range of 120-10000.

12. The formulation of claim 1, wherein the polyethylene glycol mono- or di-ether is polyethylene glycol monomethyl ether (MPEG).

13. The formulation of claim 12, wherein the mean molecular weight of the polyethylene glycol monomethyl ether (MPEG) is in the range of 350-10000.

14. The formulation of claim 13, wherein the polyethylene glycol monomethyl ether is MPEG 350-5000.

15. The formulation of claim 1, wherein the formulation comprises the polyethylene glycol mono- or di-ether in an amount of 2-15%.

16. The formulation of claim 1, wherein the formulation comprises the polyethylene glycol (PEG) or poloxamer and the polyethylene glycol mono- or di-ether in a ratio of from 10:1 to 1 :1.

17. The formulation of claim 1, comprising: (a) a pharmaceutically active agent; (b) water; (c) polyethylene glycol (PEG); and (d) polyethylene glycol monomethyl ether (MPEG).

18. The formulation of claim 1, comprising: (a) 0.1-30% pharmaceutically active agent; (b) 5-50% water; (c) 5-50% polyethylene glycol; (d) 2-15% polyethylene glycol monomethyl ether; (e) 0-70% alcohol; (f) 0-5% acid or base; (g) 0-10% penetration enhancer; and (h) 0-6% plasticizer.

19. The formulation of claim 1, comprising: (a) 0.1-30% pharmaceutically active agent; (b) 5-50% water; (c) 5-50% polyethylene glycol; (d) 2-15% polyethylene glycol monomethyl ether; (e) 0-70% alcohol; (f) 0-5% acid or base; (h) 0-1% isopropyl myristate; (i) 0-4% transcutol; and ()) 0-5% propylene glycol.

20. The formulation of claim 1, wherein the pharmaceutically active agent is an anti-fungal or anti-mycotic agent.

21. The formulation of claim 1, wherein the pharmaceutically active agent is lipophilic and/or keratinophilic.

22. The formulation of claim 1, wherein the pharmaceutically active agent is an azole, imidazole, triazole, thiazole, thiadiazole, guanidine, pyrimidine, imine, morpholine, 2-pyridone, 2-pyrimidone, allylamine, benzylamine, polyene, echinocandin, benzofuran, benzoxaborole, pyridine, or thiocarbamate.

23. The formulation of claim 22, wherein the imidazole is bifonazole, clotrimazole, econazole, fenticonazole, isoconazole, ketoconazole, miconazole, oxiconazole, tioconazole, sertaconazole, sulconazole, or a pharmaceutically acceptable salt thereof.

24. The formulation of claim 22, wherein the triazole is fluconazole, itraconazole, posaconazole, ravuconazole, terconazole, voriconazole, or a pharmaceutically acceptable salt thereof.

25. The formulation of claim 22, wherein the thiazole is a 2-amino-thiazole.

26. The formulation of claim 25, wherein the 2-amino-thiazole is abafungin or a pharmaceutically acceptable salt thereof.

27. The formulation of claim 22, wherein the guanidine is an arylguanidine.

28. The formulation of claim 27, wherein the arylguanidine is abafungin or a pharmaceutically acceptable salt thereof.

29. The formulation of claim 22, wherein the pyrimidine is a 2-pyrimidinimine.

30. The formulation of claim 29, wherein the 2-pyrimidinimine is abafungin or a pharmaceutically acceptable salt thereof.

31. The formulation of claim 22, wherein the imine is a 2-pyrimidinimine.

32. The formulation of claim 31, wherein the 2-pyrimidinimine is abafungin or a pharmaceutically acceptable salt thereof.

33. The formulation of claim 22, wherein the morpholine is amorolfine or a pharmaceutically acceptable salt thereof.

34. The formulation of claim 22, wherein the 2-pyridone is ciclopirox or a pharmaceutically acceptable salt thereof.

35. The formulation of claim 22, wherein the 2-pyrimidone is flucytosine or a pharmaceutically acceptable salt thereof.

36. The formulation of claim 22, wherein the allylamine is terbinafine, naftifine, or a pharmaceutically acceptable salt thereof.

37. The formulation of claim 22, wherein the benzylamine is butenafine or a pharmaceutically acceptable salt thereof.

38. The formulation of claim 22, wherein the polyene is amphotericin B, nystatin, pimaricin (also called natamycin), or a pharmaceutically acceptable salt thereof.

39. The formulation of claim 22, wherein the echinocandin is caspofungin, micafungin, anidulafungin, or a pharmaceutically acceptable salt thereof.

40. The formulation of claim 1, wherein the pharmaceutically active agent is abafungin, ciclopirox olamine, terbinafine hydrochloride, or amorolfine.

41. The formulation of claim 1, wherein the pharmaceutically active agent is abafungin or a pharmaceutically acceptable salt thereof.

42. The formulation of claim 1, wherein the pharmaceutically active agent is substantially dissolved in the formulation.

43. The formulation of claim 1, further comprising an alcohol.

44. The formulation of claim 43, wherein the alcohol is 2-propanol or ethanol.

45. The formulation of claim 1, further comprising an acid or a base.

46. The formulation of claim 45, wherein the acid is formic acid.

47. The formulation of claim 1, further comprising a penetration enhancer and/or a plasticizer.

48. The formulation of claim 1, further comprising isopropyl myristate.

49. The formulation of claim 1, further comprising a penetration enhancer.

50. The formulation of claim 49, wherein the penetration enhancer is transcutol.

51. The formulation of claim 1, further comprising propylene glycol.

52. The formulation of claim 1, wherein the formulation has a viscosity of at least 1100 mPas.

53. The formulation of claim 1, wherein the formulation is a hydrophilic water-based gel.

54. The formulation of claim 1 formulated for topical application.

55. A method of treating a disease, disorder or pathological condition of the nail, mucosa, or skin comprising administering to a subject in need thereof a formulation comprising: (a) a pharmaceutically active agent; (b) water; (c) a polyethylene glycol (PEG) or a poloxamer; and (d) a polyethylene glycol mono- or di-ether.

56. The method of claim 55, wherein the disease, disorder or pathological condition is selected from the group consisting of onychomycosis, dermatomycosis, an oral, vaginal or anal mycosis, a skin disease, a topical bacterial infection, or a topical viral infection.

57. The method of claim 55, wherein the disease, disorder or pathological condition is wound healing.

58. The method of claim 55, wherein the formulation is administered to a nail of the subject.

59. The method of claim 58, wherein the pharmaceutically active agent penetrates into the subject's nail and nail matrix by penetrating through the nail and through the skin surrounding the nail.

60. A method of treating onychomycosis according to claim 56, comprising applying the formulation to the nail of a subject suffering from onychomycosis.

61. A method of treating dermatomycosis according to claim 56, comprising applying the formulation to the skin of a subject suffering from dermatomycosis.

62. A method of treating an oral, vaginal or anal mycosis according to claim 56, comprising applying the formulation to a subject suffering from the oral, vaginal or anal mycosis.

63. A method of treating a skin disease according to claim 56, comprising topically applying the formulation to the skin of a subject suffering from the skin disease.

64. The method of claim 55, wherein the disease, disorder or pathological condition is a topical bacterial infection or a topical viral infection, comprising topically applying the formulation to a subject suffering from the topical infection.

65. The method of claim 57, comprising topically applying the formulation to the wound of a subject.

66. The method of claim 55, wherein the subject is a human.

67. A method of preparing the formulation of claim 1, comprising the steps of: (a) dissolving the pharmaceutically active agent and, if present, the acid or base in water; (b) adding the polyethylene glycol or poloxamer, the polyethylene glycol mono- or di-ether and, if present, the alcohol, the penetration enhancer and the plasticizer to the solution; and (c) stirring the mixture until a hydrophilic gel is obtained.

68. The method of claim 67, wherein the pharmaceutically active agent can be protonated and an acid is used in step (a).

69. The method of claim 68, wherein the pharmaceutically active agent is abafungin.

70. The method of claim 67, wherein the pharmaceutically active agent can be deprotonated and a base is used in step (a).
